(19) United States
(12) Patent Application Publication
(10) Pub. No.: US 2012/0288558 A1
(43) Pub. Date: Nov. 15, 2012

(54) METHOD FOR ADMINISTRATION OF PEGYLATED LIPOSOMAL DOXORUBICIN

(76) Inventor: Alberto A. Gabizon, Jerusalem (IL)

(21) Appl. No.: 13/552,433

(22) Filed: Jul. 18, 2012

Related U.S. Application Data
(63) Continuation of application No. 12/517,864, filed on Jun. 5, 2009.
(60) Provisional application No. 60/870,949, filed on Dec. 20, 2006.

(51) Int. Cl. A61K 9/127; A61P 35/00; A61K 31/704

(52) U.S. Cl. 424/450; 514/34

(57) ABSTRACT

An embodiment of the present invention comprises a method of treating malignancies in a subject in need of treatment comprising administering to the subject a high loading dose of a pegylated liposomal doxorubicin (PLD) in an initial cycle, followed by a reduced dose in a second cycle, wherein the second cycle reduced dose is in the range of 20% to 50%, preferably 50%, of the initial loading dose, and thereafter one or more maintenance doses in further cycles. The interval between dose cycles is in the range of about three-to-four weeks, preferably about four weeks. The initial loading dose is in the range of between the maximum tolerated dose (MTD) and the recommended dose, preferably the MTD (for instance, in the range of about 70 mg/m2 to 50 mg/m2, preferably 60 mg/m2). The one or more maintenance doses are in the range of about 40 mg/m2 to 50 mg/m2, preferably 45 mg/m2).

### FIELD OF THE INVENTION

The present invention is directed to methods of cancer treatment using pegylated liposomal doxorubicin (PLD).

### BACKGROUND OF THE INVENTION

The anthracycline antibiotic doxorubicin has a broad spectrum of antineoplastic action and a correspondingly widespread degree of clinical use. In addition to its role in the treatment of breast cancer, doxorubicin is indicated in the treatment of Hodgkin's Disease and non-Hodgkin's lymphoma, hepatocellular and gastric carcinoma, small cell cancer of the lung, soft tissue and bone sarcomas, as well as cancer of the ovary, bladder and thyroid. Unfortunately, toxicity often limits the therapeutic activity of doxorubicin and may preclude adequate dosing.

Pegylated liposomal doxorubicin (PLD) (marketed under the tradenames DOXIL® and CAELYX®) is a doxorubicin formulation in which the drug is encapsulated in liposomes (STEALTH Liposomes®). It was designed to enhance the efficacy and reduce the dose-limiting toxicities of doxorubicin by altering the plasma pharmacokinetics and tissue distribution of the drug. Preclinical results show that PLD prolongs the systemic circulation of doxorubicin, leading to higher concentrations of the drug in tumors and resulting in a reduction in tumor mass and prolonged survival.

DOXIL®/CAELYX® was granted market clearance in 1995 by the US Food and Drug Administration (FDA) for the use in treatment of AIDS-KS in patients with disease that has progressed on prior combination chemotherapy and who are intolerant to such therapy. In 1996 it was granted market clearance by the European Union's comission for Proprietary Medicinal Products for the same indication. In 1999, DOXIL®/CAELYX® was granted US market clearance for the use in the treatment of metastatic carcinoma of the ovary in patients with disease that is refractory to paclitaxel- and platinum-based chemotherapy regimens. In Jan. 2003, the European Commission of the European Union has granted centralized marketing authorization to DOXIL®/CAELYX® as monotherapy for metastatic breast cancer in patients who are at increased cardiac risk.

The pharmacokinetic (PK) advantage of PLD is the enhancement of tumor exposure to doxorubicin as a result of the accumulation of stealth liposomes in tumors, as demonstrated in animal models and in human cancer. The pharmacokinetics of PLD is characterized by long-circulation time and minimal drug leakage (<5%) from circulating liposomes (1). The clearance of the liposomal carrier is the primary determinant of the pharmacokinetics of PLD, given the negligible rate of drug leakage (1).

The impact of dose on drug accumulation in the tumor has been only studied in animals prior to the clinical study of the present invention. These animal studies demonstrated that dose escalation results in a saturation of PLD clearance and disproportional increase of the amount of liposomal drug accumulation in tumor. In preclinical models, prior treatment with PLD has been shown to cause a delay in clearance of drug-free liposomes, indicating damage or saturation of the reticulo-endothelial system (RES) (2). This temporary inhibition of RES-mediated liposome clearance is caused specifically by PLD, and is not observed with free doxorubicin (2), or with drug-free pegylated liposomes for which clearance is dose-independent over a wide dose range (3).

In human studies, a trend to longer half-life and slower clearance has been observed in patients receiving higher doses compared with those receiving lower doses. The available data, however, is insufficient to distinguish between interpatient variability or a phenomenon of clearance saturation due to dose-dependent pharmacokinetics. The results of various PK studies with PLD point to half-lives in the range of 50-55 hours for dose levels of 10-20 mg/m² in AIDS-related Kaposi's sarcoma patients (4), and around 60-80 hours for dose levels of 35-70 mg/m² in solid tumor patients (1). In pediatric patients receiving 40-70 mg/m², the half life is significantly shorter averaging 36 hours (5). One study (4) examined the PK of PLD when the dose is escalated in the same patient population from 10 to 20 mg/m², and found not evidence of dose-dependent PK. Yet, no study has addressed the PK effects of a change in dose and repeated treatment with PLD in the dose range of solid tumors (30-60 mg/m²) with intra-individual comparisons.

PLD has major advantages over doxorubicin and other anthracyclines with regard to important toxicity parameters such as cardiomyopathy (6-10), myelosuppression, and alopecia (reviewed in (8)). However, treatment with PLD is associated with a high incidence of stomatitis and palmar-plantar erythema (PPE, also known as hand-foot syndrome) (8, 11, 12). Indeed, skin toxicity, in the form of PPE, and stomatitis are the dose-limiting toxicities of PLD (12). Although not life-threatening, PPE is problematic to control and/or foresee since it usually occurs after cumulative damage to the skin from two or more courses of PLD. Stomatitis is generally correlated with peak dose level (13, 14). Skin toxicity correlates with dose interval, dose intensity, and T½ (half-life) of PLD (12, 13, 15). Skin toxicity of PLD tends to manifest after 2 or more cycles of treatment (11, 12), hinting at a complex PK-PD relationship.

Thus, there is a great need for a PLD protocol which would optimize the beneficial treatment effects of PLD while minimizing or eliminating the incidence of stomatitis and PPE.

### SUMMARY OF THE INVENTION

An embodiment of the present invention comprises a method of treating malignancies in a subject in need of treatment comprising administering to the subject a high loading dose of a pegylated liposomal doxorubicin (PLD) in an initial cycle, followed by a reduced dose in a second cycle, wherein the second cycle reduced dose is in the range of 20% to 50%, preferably 50%, of the initial loading dose, and thereafter one or more maintenance doses in further cycles. The interval between dose cycles is in the range of about three-to-four weeks, preferably about four weeks. The initial loading dose is in the range of between the maximum tolerated dose (MTD) and the recommended dose, preferably the MTD (for instance, in the range of about 70 mg/m² to 50 mg/m², preferably 60 mg/m²). The one or more maintenance doses are in the range of about 40 mg/m² to 50 mg/m², preferably 45 mg/m²).

Other features and advantages of the invention will be apparent from the following detailed description of the invention and from the claims.

### BRIEF DESCRIPTION OF THE FIGURES

FIG. 1 illustrates the design of the clinical trial conducted in the present invention.

FIG. 2 illustrates the PK parameters of PLD by dose and cycle (panels A-E). A: Cmax; B: T½; C: AUC; D: CL; E: Vss.

FIG. 3 illustrates the PLD plasma clearance curve as fraction of injected dose comparing 30 mg/m² to 60 mg/m² dose level (panel A), and 1^{st} to 3^{rd} cycle (panel B). Slope comparison: A, not significant (n=13); B, p=0.0040 (n=12)

### DETAILED DESCRIPTION OF THE INVENTION

Prior to the studies of the present invention, it had been assumed that the pharmacokinetic properties of PLD are independent of dose. Data from in-vivo animal systems, however, suggests that this assumption may be incorrect and that PLD indeed have saturation kinetics with substantially prolonged clearance in addition to increased tumor uptake at higher doses.

Given that there are dose response relationships for both anti-tumor and adverse effects, these pharmacokinetic considerations may have implications for optimal therapeutic dosing. For purposes of the present invention, a clinical study aimed at examining the dose and cycle dependency of PLD PK was carried out. The study evaluated the effect of PLD dose on its PK properties in order to determine whether a dose increase causes saturation of clearance, i.e. is the PK of PLD dose dependent.

### Patients and Methods

Study Design:

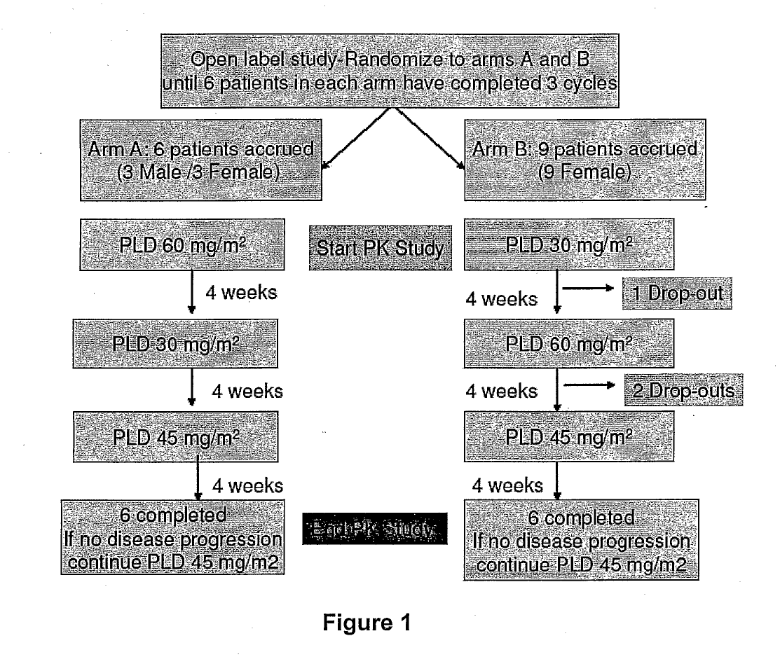

As seen in FIG. 1, patients with various solid tumors were randomized to two arms of treatment (A and B) in an open-label study design. Group A received PLD at 60 mg/m² in the 1^{st} cycle, 30 mg/m² in the 2^{nd} cycle, and 45 mg/m² in the 3^{rd} cycle. Group B received PLD at 30 mg/m² in the 1^{st} cycle, 60 mg/m² in the 2^{nd} cycle, and 45 mg/m² in the 3^{rd} cycle. All cycles were given at 4-week intervals. The aim was to have at least 6 patients per arm completing all 3 cycles. Because 3 patients dropped out of the study before completing 3 cycles, a total of 15 patients were recruited to get 12 fully evaluable patients. This study was designed to obtain information on the effect of a two-fold change in the dose level as well as on the effect of repeated cyles of therapy. To balance the effect of an increasing versus decreasing dose along time on study, patients were randomized into mirror groups A and B. To avoid the confounding factor of inter-patient variability, the approach here was based on intra-patient comparison of PK data using the paired t test for statistical analysis. The dose levels chosen were based on prior clinical experience with PLD in solid tumors to ensure that most patients could complete the study without dose reductions or delays. This specific design enabled to maximize the information and statistical value obtained from a small group of 12 patients.

The study protocol was approved by the Institutional Review Board of the Shaare Zedek Medical Center and required signed witnessed consent. For randomization, a total of six A and six B ballots was used as pool. In case of a patient drop-out the corresponding ballot was returned to the pool.

Dose, Administration and Treatment Schedule:

The study drug, DOXIL®/CAELYX®, was supplied by Ortho Biotech L.P. in sterile vials, each containing 20 mg doxorubicin hydrochloride at a concentration of 2.0 mg/mL. Storage and handling was in accordance with the Labeling Instructions for Drug Storage and Administration of DOXIL®/CAELYX®. DOXIL®/CAELYX® was diluted in 500 ml 5% Dextrose Injection, USP (D₅W) in accordance with the Labeling Instructions for Drug Storage and Administration of and once diluted it kept refrigerated at 2° to 8° C. and administered within 24 hours of mixing. Administration of DOXIL®/CAELYX® was by infusion through a peripheral vein or a central line at a rate of 8-10 ml/minute in accordance with the labeling. To avoid acute reactions to DOXIL®/CAELYX® infusion, treatment was started at 1/10 of the final infusion rate. If the patient had no signs and symptomes of reaction after 10-15 minutes, the rate of infusion was gradually increased to the target infusion rate.

Premedication:

Pre-medication was administered as follows: On day 1 granisetron 3 mg (or ondansetron 8 mg) IV will be given within 30 minutes prior to treatment (according to its datasheet). Patients with acute symptomes of nausea and/or vomiting, will also receive premedication of dexamethasone 8 mg IV. All other antiemetic therapy will be given depending on how patients tolerate the infusion and physician discretion.

Plasma Sampling:

Blood (3-5 ml) was withdrawn into vacuum sealed K-EDTA containing tubes at the following time points: Pre-infusion, 1 h, 24 h, between 72-96 h, 7±1 days, 14±1 days, 21±1 days, and 28±1 days after infusion. Plasma was separated by centrfugation and stored at −20° C. until testing.

Analysis of PLD Concentration:

Plasma levels of doxorubicin were analyzed by HPLC-fluorimetry following the method of Chin et al. (16) with minor modifications. For extraction of doxorubicin, 1 μg daunorubicin was added as internal standard to 200 μl of plasma and the mixture was vortexed. We then added 20 μl of 3% (v/v) Triton X-100, followed by 20 μl of 65% (w/v) 5-sulfosalycilic acid. After each addition, the sample was vortexed for 10 sec. The next step was centrifugation of the samples for 5 min at 20,000 g. The supernatants were harvested, and 35 μl of 3 M sodium acetate was added to each sample, followed by filtration through 0.22 μm-pore membranes. The filtered samples were injected (100 μl/injection) with an automatic injector into an isocratic HPLC system with a mobile phase consisting of 35% acetonitrile/65% DDW containing 10 mg/L desipramine at pH 2.5, using an Econosphere C8-5 μm column (length 150 mm, internal diameter 4.6 mm), and a flow rate of 2 ml/min, for a total run time of 10 min per sample. Doxorubicin (retention time: 2.60 min) and daunorubicin (retention time: 3.60 min) were detected with a fluorescence detector at ex:470/em:590 nm wavelength. The concentration of doxorubicin was calculated based on the relative peak areas of doxorubicin and daunorubicin, the internal standard. This system and method were able to detect doxorubicin within a range of 10 ng to 5 μg. Since the extraction method detroys the liposomes, the drug measured here represents the total amount of drug in the plasma including the liposomal fraction, protein-bound fraction, and free fraction. However, since data from various studies (4, 17, 18) indicate that >95% of the doxorubicin measured in plasma is liposome bound, the results presented here can be considered representative of the PK of PLD itself.

PK analysis was done by non-compartmental method using PK Solutions™ software (Summit Research Services, Montrose, Colo.). The following parameters were obtained: Cmax (peak plasma concentration, Y-intercept), terminal half-life (T½), area under the curve from zero to infinity (AUG∞), clearance (CL, dose/AUC), and volume of distribution at steady state (Vss, dose-AUMC/AUC²). Statistical analysis (paired t test) was done using Prism software (Graphpad, San Diego, Calif.).

### Results

### Patient Characteristics, Toxicity, and Treatment Outcome:

Fifteen patients suffering from various malignant solid tumors were accrued to this study (Table 1).

TABLE 1
Patient Characteristics

| Total number of patients | 15 |
|---|---|
| Sex: Male/Female | 3/12 |
| Age: median (range) | 61 (33-78) |
| Type of Cancer: | N Patients |
| Soft tissue sarcoma | 4 |
| Breast carcinoma | 3 |
| Ovarian carcinoma | 3 |
| Stomach carcinoma | 2 |
| Peritoneal (1ary) carcinoma | 1 |
| Prostate carcinoma | 1 |
| Thymoma (epithelial) | 1 |
| Prior chemotherapy, Yes/No | 14/1 |
| For Metastatic Disease | 11 |
| As Adjuvant only | 3 |
| Prior anthracyclines Yes/No | 6/9 |
| Median ECOG P.S. | 1 (0-2) |
| ECOG-0/1/2 | 6/5/4 |
| Median No of cycles, (range) | 9 (1-22+) |

Females were over-represented, but this goes along with the clinical use of PLD which is mainly in ovarian and breast cancers. Patient accrual began on October 2004 and was completed within 12 months. Three patients did not complete the 3 study cycles (see FIG. 1), one after 1 cycle because of frank disease progression and two after 2 cycles because of disease progression and toxicity (see below for details).

### Adverse Events:

All adverse events that occur at any time during the study period as defined were reported. Each patient was evaluated at each patient visit during the study for any new or continuing symptoms. Any symptoms changing in character or in intensity were noted. Any clinically significant adverse event reported by the patient or caregiver, or noted by the investigator or study coordinator was recorded. The intensity of the adverse event was evaluated, relationship of the adverse event to the PLD study drug was determined. Intensity of the adverse event will be evaluated using the following criteria: Mild (Grade 1): The patient is aware of the sign or symptom but tolerates it easily. The event is of little concern to the patient and of little clinical significance. The event is not expected to have any effect on the patient's overall health or well-being. Moderate (Grade 2): The patient has discomfort enough to cause interference with or change in usual activities. The event is of some concern to the patient's health or well being and may require medical intervention and/or close follow-up. Severe (Grade 3): The adverse event interferes considerably with the patient's usual activities. The event is of definite concern to the patient and/or poses substantial risk to the patient's health or well-being. The event is likely to require medical intervention and/or close follow-up and may be incapacitating or life-threatening. Hospitalization and treatment may be required. Life-Threatening (Grade 4): The patient is incapacitated. The event poses substantial risk to the patient's immediate health or well-being.

Treatment was generally well tolerated except for 3 heavily pretreated patients with advanced disease in whom all the severe toxicities seen in this study were clustered. One patient with recurrent carcinoma of esophagus after chemoradiotherapy developed mucositis (esophagitis) grade 3 after a first course of PLD at 60 mg/m² and was treated ambulatorily with intravenous fluids. A second patient with heavily pretreated metastatic breast cancer developed neutropenic fever and stomatitis grade 3 requiring hospitalization after 2 courses of PLD, (30 mg/m², followed by 60 mg/m²). Although she recovered from toxicity within 7-10 days, she was not further treated with PLD given the appearance of obstructive jaundice and evidence of progressive disease. A third patient with pretreated metastatic gastric cancer and severe ascites developed neutropenia grade 4 and stomatitis grade 4 after 2 courses of PLD (30 mg/m² followed by 60 mg/m²). PLD was discontinued as she recovered only partially remaining bedridden and requiring protracted hospitalization further complicated by evidence of progressive disease. Both of these cases also suffered form PPE grade 3. Other cases of PPE were of lesser severity and did not affect the course of treatment.

There was no evidence of cardiac toxicity, neither clinical nor radio-angiocardiographic (MUGA scan) with the maximal cumulative dose reaching in one of the patients 925 mg/m² by October 2006. Moderate to severe hair loss (grade 2) was observed in only one patient. All other patients had none or minimal hair loss.

With a minimal follow-up of 1 year by October 2006, the median time to disease progression is 8 months (range: 1-24+). Median survival has not yet been reached (8 alive, 7 dead) and stands at 16+ months (range: 1-24+). The median number of cycles given per patient is 9 (range: 1-22+). Several durable (>1 yr-long) stabilizations with or without objective anti-tumor responses were observed in sarcoma (2), ovarian (1), breast (1), and prostate (1) carcinoma patients.

PK Results:

Table 2 presents a summary of dose and cycle comparisons with the numerical values of PK parameters.

TABLE 2
Summary of Dose and Cycle Comparisons: Mean (SEM)

| PK | | | | | |
|---|---|---|---|---|---|
| Parameter | 30 mg/m² | 60 mg/m² | 1^{st} Cycle | 2^{nd} Cycle | 3^{rd} Cycle |
| Cmax/mg | 413 (24) | 413 (32) | 406 (26) | 420 (31) | 475 (29) |
| dose (μg/L) | | | | | |
| T½ (hr) | 76 (4.9) | 83 (7.0) | 73 (5.3) | 86 (6.4)* | 87 (6.5)* |
| AUC∞/mg | 49 (4.1) | 53 (5.5) | 46 (3.8) | 56 (5.4)* | 66 (6.1)* |
| dose | | | | | |
| (mg*hr/L) | | | | | |
| CL (mL/hr) | 22 (1.8) | 21 (2.5) | 24 (2.2) | 20 (1.9)* | 16 (1.5)* |
| Vss (L) | 2.5 (0.2) | 2.5 (0.2) | 2.5 (0.2) | 2.3 (0.2) | 2.0 (0.1)* |

*Significantly different from 1st cycle. None of the dose comparisons were significant. Statistical analysis of 1st vs. 3rd cycle:
Cmax, not significant;
T½, p = 0.0127;
AUC, p = 0.0005;
CL, p = 0.0003;
Vss, p = 0.0191.

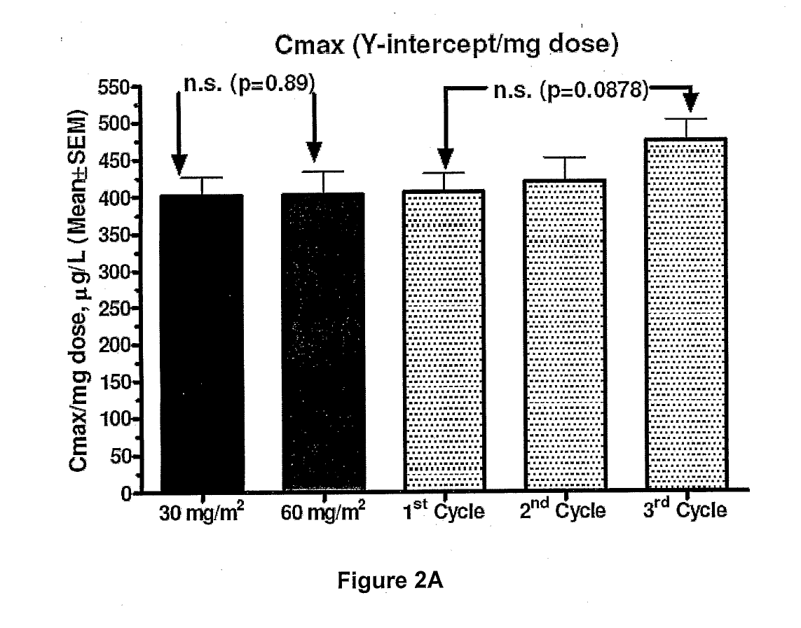

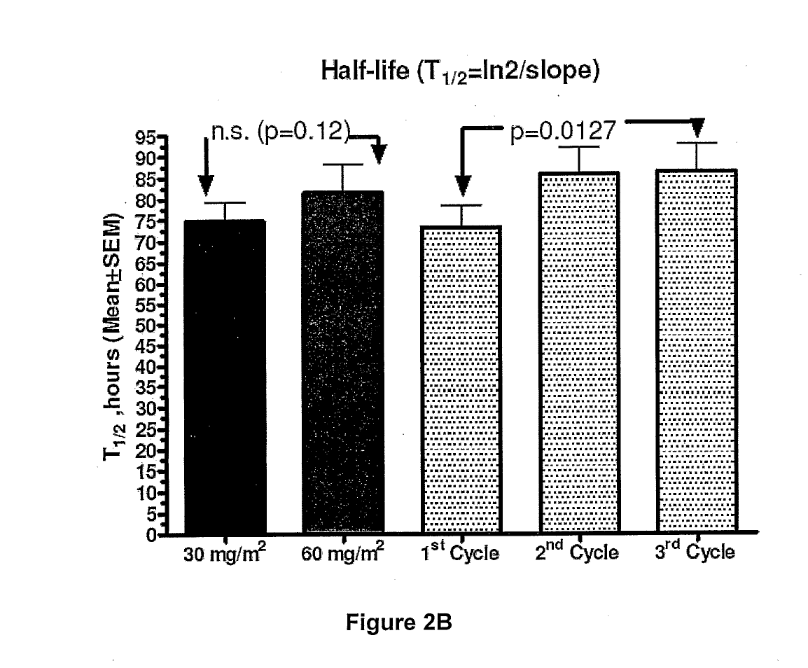

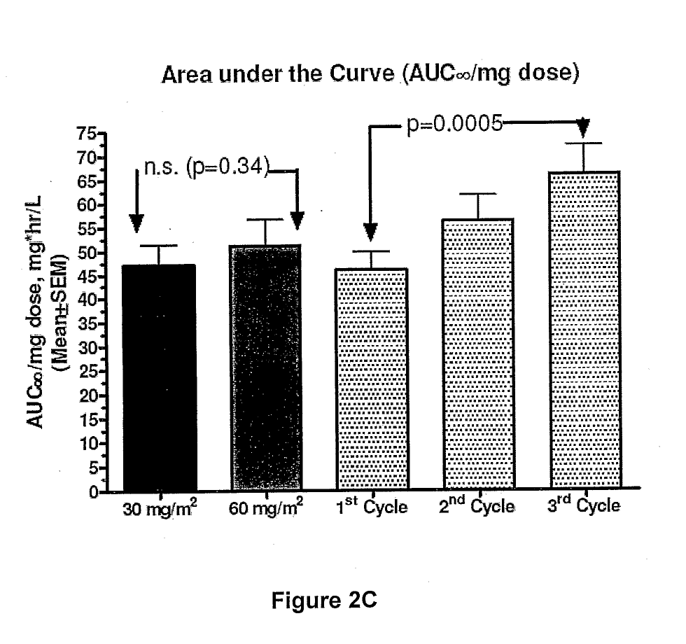

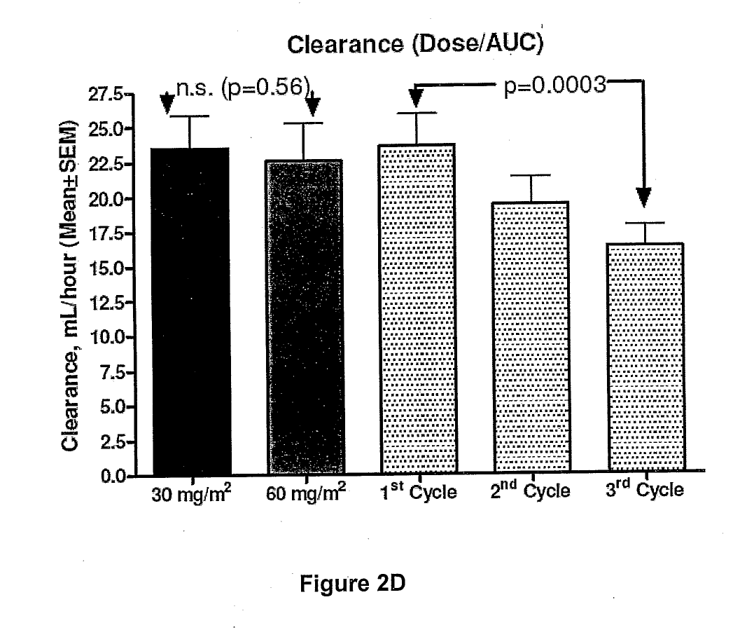

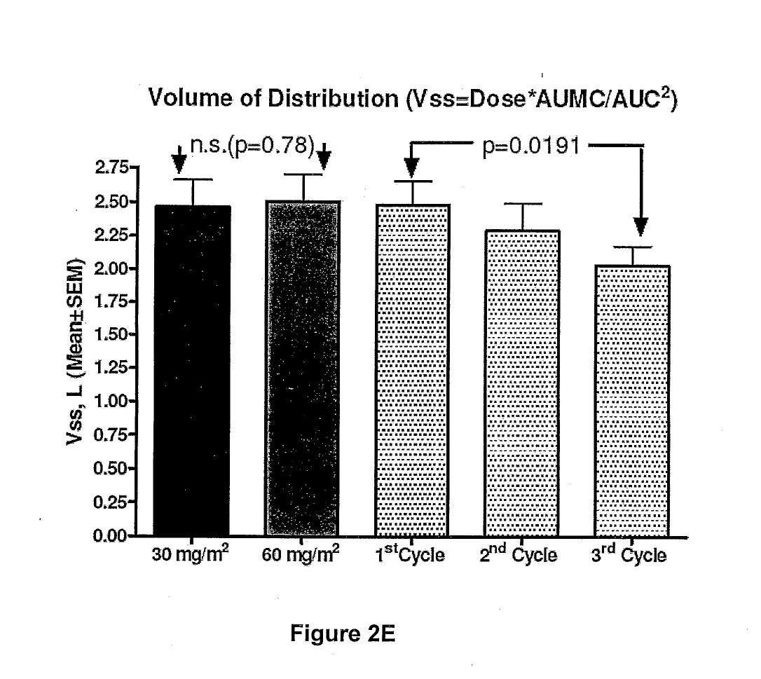

FIGS. 2A through 2E illustrates the PK parameters determined by dose and cycle. A: Cmax; B: T½; C: AUC; D: CL; E: Vss.

When the 30 and 60 mg/m² dose level are compared, there was no significant change in any of the PK parameters analyzed −Cmax and AUC both normalized per mg dose, T½, CL, and Vss (see Table 2 and FIG. 2). In contrast, there is a significant increase of dose-normalized AUC values and a correspondingly significant decrease of CL values when comparing the 1^{st} cycle of treatment to the 2^{nd} cycle and more so to the 3^{rd} cycle (see Table 2 and FIGS. 2C-2D). Note that a 44% increase in AUC occurs when the 1^{st} and 3^{rd} treatment cycles are compared, pointing to a major potential increase in patient exposure to drug by merely retreating the patient without increasing the dose. Terminal T½ was also significantly prolonged when the 1^{st} and 3^{rd} cycles are compared (see Table 2 and FIG. 2B), while other parameters (Cmax, Vss) were affected to a much lesser extent—nonsignificant increase (+17%) of Cmax (see Table 2 and FIG. 2A), and significant decrease (−20%) of Vss. (see Table 2 and FIG. 2E).

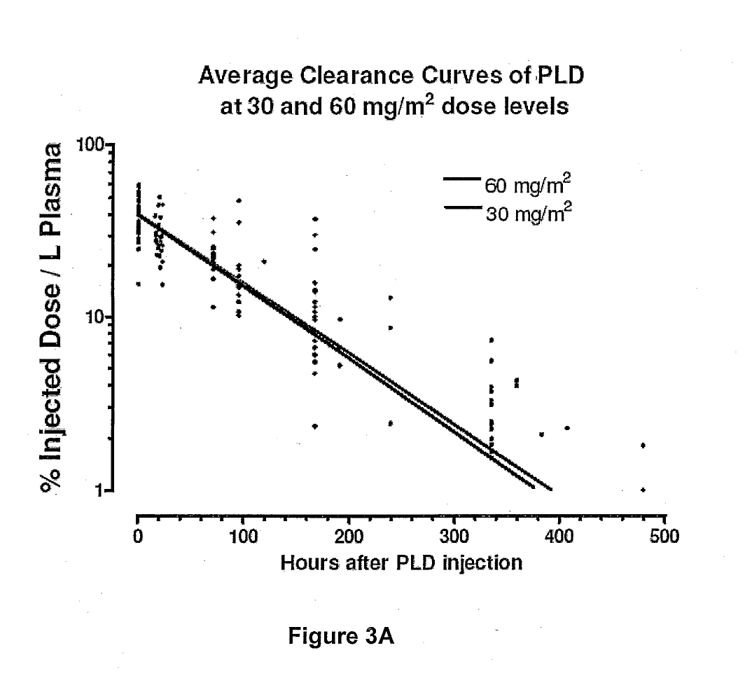

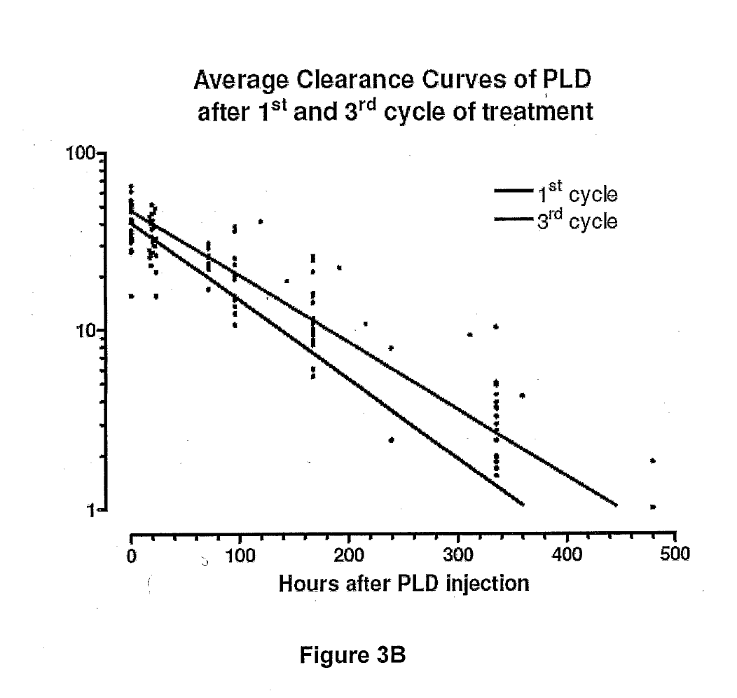

To compare the plasma clearance curves for all patients examined according to dose level (30 or 60 mg/m²) or cycle number (1^{st} vs. 3^{rd} cycle), we transformed the plasma concentration values from μg/ml plasma to injected dose per liter plasma, and performed regression analysis using the equation, Concentration=A*e^{-B*Time}, where A (Y-intercept) is the Cmax average and B (slope) is the average of the elimination rate constant of each dose/cycle group tested. FIG. 3 illustrates the PLD plasma clearance curve as fraction of injected dose comparing 30 mg/m² to 60 mg/m² dose level (panel A), and 1^{st} to 3^{rd} cycle (panel B). Slope comparison: A, not significant (n=13); B, p=0.0040 (n=12)

As seen in FIG. 3, the resulting curves clearly underscore that, while clearance is not affected within the dose range 30-60 mg/m², a substantial retardation in clearance is observed with retreatment when the 1^{st} and 3^{rd} cycles of PLD are compared. This is underscored by a statistically significant difference when comparing the slopes of the curves of FIG. 3B (p=0.0040).

### Discussion

While free drugs are mainly handled by hepatic and/o renal clearance, nanoparticles such as liposomes are mainly cleared by the RES. Polyethylene-glycol (PEG) coating of liposomes protects liposomes from opsonization and delays their clearance from circulation, preventing the rapid and massive RES uptake seen after injection of non-pegylated liposomes (19). Prolonged stay in circulation enables liposomes to reach in greater amounts tissues with transient or inherent increase in vascular permeability such as specific skin areas and tumors (20, 21), but ultimately Kupffer cells, spleen, and bone marrow macrophages are the major liposome destination (22). Therefore, RES-mediated clearance plays a major role in determining the PK of formulations such as PLD, and factors affecting RES function will have an impact on liposomal drug clearance. Unfortunately, there are no clinical tests of RES function that could predict the clearance of particulate carriers. However, preclinical findings indicating temporary depression of RES activity after administration of PLD as measured by bacterial clearance (23) or by clearance of an additional dose of radiolabeled liposomes (2).

The dose range tested, 30 to 60 mg/m², is most relevant since it covers the spectrum of dose used in the treatment of patients with solid tumors (8). By dividing the patients in 2 groups with reversed order of treatment (30→60 mg/m² and 60→30 mg/m²), we wished to neutralize any variability due to cycle number rather than to dose change. In addition, by adding a 3^{rd} cycle of treatment at the same dose (45 mg/m²) to all patients, we could obtain reliable information on the PLD PK along 3 cycles of treatment with a balanced dose distribution and maximize the value of the study. Statistical analysis using the paired t test ensures a simple and powerful method to detect significance for both dose and cycle effects in such a small patient population.

We were not able to detect any significant change in clearance rate of PLD when 30 mg/m² and 60 mg/m² doses were compared, leading to the conclusion that the PK of PLD is dose-independent. This is consistent with results from a previous study in metastatic breast cancer patients (13) in which a minimal and non-significant change in clearance was observed when doses of 35 and 70 mg/m² were compared, albeit across different patient cohorts. This would suggest, in principle, a lack of RES saturation after PLD treatment. However, our finding of an inhibition of clearance upon retreatment with PLD indicate that the PK is cycle-dependent and that prior exposure to PLD is likely to be followed by inhibition of RES-mediated liposome clearance.

These seemingly conflicting findings can be reconciled by a simple explanation: liposome processing, and intra-cellular release of doxorubicin are relatively slow processes resulting in a lag phase between PLD exposure and toxicity manifested by inhibition of RES uptake of liposomes. Thus, dose escalation of PLD within the therapeutic dose range does not cause significant RES saturation, but, nevertheless, it results in a delayed damage to the RES which manifests as slower liposome clearance upon subsequent treatments. This effect may account for the delayed skin toxicity of PLD. Since AUC values are well correlated with dose of PLD (13), this would amount to ˜40-50% increase in patient exposure to drug when going from 1^{st} to 3^{rd} cycle without changing the dose, according to the results of our current study.

To avoid delayed toxicity, clinicians often refrain from using the maximum tolerated dose (MTD) (currently 60-mg/m² q4w for DOXIL®/CAELYX®) (12) and the recommended dose (currently 50 mg/m² q4w DOXIL®/CAELYX®) (24) of PLD. In fact, a dose of 40 mg/m² q4w has been proposed as a convenient starting dose for treating recurrent ovarian cancer while avoiding skin toxicity (25). This is so despite evidence in Kaposi's sarcoma (4) and preclinical models (2, 26) for a correlation of average Cmax and/or peak dose level with therapeutic efficacy.

The present invention presents a new method for minimizing the risk of delayed toxicity and avoiding the unnecessary reduction of the starting dose of PLD, by the use of a high loading dose in the initial cycle, followed by a reduced dose in the second cycle and thereafter by one or more lower maintenance doses in further cycles.

An embodiment of the present invention comprises a method of treating malignancies in a subject in need of treatment comprising administering to the subject a high loading dose of a pegylated liposomal doxorubicin (PLD) in an initial cycle, followed by a reduced dose in a second cycle, wherein the second cycle reduced dose is in the range of 20% to 50%, preferably 50%, of the initial loading dose, and thereafter one or more maintenance doses in further cycles. The interval between dose cycles is in the range of about three-to-four weeks, preferably about four weeks. The initial loading dose is in the range of between the maximum tolerated dose (MTD) and the recommended dose, preferably the MTD (for instance, in the range of about 70 mg/m² to 50 mg/m², preferably 60 mg/m²). The one or more maintenance doses are in the range of about 40 mg/m² to 50 mg/m², preferably 45 mg/m²).

In an embodiment of the present invention, the malignancies are solid tumor malignancies, for instance, adrenocortical carcinoma, bladder carcinoma, breast carcinoma, colorectal carcinoma, desmoid tumors, desmoplastic small round cell tumor, endocrine tumors, endometrial carcinoma, epithelial carcinomas, Ewing sarcoma family tumors, germ cell tumors (solid tumor), head and neck carcinoma, hepatoblastoma, hepatocellular carcinoma, lung carcinoma, melanoma, nasopharyngeal carcinoma, neuroblastoma, non-rhabdomyosarcoma soft tissue sarcoma (NRSTS), osteosarcoma, ovarian carcinoma, pancreatic carcinoma, peripheral primitive neuroectodermal tumor (PPNET), peritoneal carcinoma, prostate carcinoma, retinoblastoma, rhabdomyosarcoma, sarcomas, soft tissue sarcoma, stomach carcinoma, thymoma (epithelial), uterine carcinoma, and Wilms tumor.

In an embodiment of the present invention, the malignancies are hematological malignancies, such as leukemias, lymphomas (non Hodgkin's lymphoma), Hodgkin's disease (also called Hodgkin's lymphoma), and myeloma for instance, acute lymphocytic leukemia (ALL), acute myeloid leukemia (AML), acute promyelocytic leukemia (APL), chronic lymphocytic leukemia (CLL), chronic myeloid leukemia (CML), chronic neutrophilic leukemia (CNL), acute undifferentiated leukemia (AUL), anaplastic large cell lymphoma (ALCL), prolymphocytic leukemia (PML), juvenile myelomonocyctic leukemia (JMML), adult T cell ALL, AML with trilineage myelodysplasia (AML/TMDS), mixed lineage leukemia (MLL), myelodysplastic syndromes (MDSs), myeloproliferative disorders (MPD), multiple myeloma, (MM) and myeloid sarcoma.

The method of present invention balances the actual dose exposure of patients to PLD when going from first to subsequent cycles. It would allow administering an optimal dose for anti-tumor response, and avoiding occurrence of toxicity to dictate dose reduction.

### REFERENCES

1. Gabizon A, Shmeeda H, Barenholz Y. Pharmacokinetics of pegylated liposomal Doxorubicin: review of animal and human studies. Clin Pharmacokinet 2003; 42(5):419-36.
2. Gabizon A, Tzemach D, Mak L, Bronstein M, Horowitz A T. Dose dependency of pharmacokinetics and therapeutic efficacy of pegylated liposomal doxorubicin (DOXIL) in murine models. J Drug Target 2002; 10(7):539-48.
3. Allen T M, Hansen C. Pharmacokinetics of stealth versus conventional liposomes: effect of dose. Biochim Biophys Acta 1991; 1068(2):133-41.
4. Amantea M A, Forrest A, Northfelt D W, Mamelok R. Population pharmacokinetics and pharmacodynamics of pegylated-liposomal doxorubicin in patients with AIDS-related Kaposi's sarcoma. Clin Pharmacol Ther 1997; 61(3):301-11.
5. Marina N M, Cochrane D, Harney E, Zomorodi K, Blaney S, Winick N, et al. Dose escalation and pharmacokinetics of pegylated liposomal doxorubicin (Doxil) in children with solid tumors: a pediatric oncology group study. Clin Cancer Res 2002; 8(2):413-8.
6. Safra T, Muggia F, Jeffers S, Tsao-Wei D D, Groshen S, Lyass O, et al. Pegylated liposomal doxorubicin (doxil): reduced clinical cardiotoxicity in patients reaching or exceeding cumulative doses of 500 mg/m2. Ann Oncol 2000; 11(8):1029-33.
7. Gabizon A A, Lyass O, Berry G J, Wildgust M. Cardiac safety of pegylated liposomal doxorubicin (Doxil/Caelyx) demonstrated by endomyocardial biopsy in patients with advanced malignancies. Cancer Invest 2004; 22(5):663-9.
8. Alberts D S, Muggia F M, Carmichael J, Winer E P, Jahanzeb M, Venook A P, et al. Efficacy and safety of liposomal anthracyclines in phase I/II clinical trials. Semin Oncol 2004; 31(6 Suppl 13):53-90.
9. O'Brien M E, Wigler N, Inbar M, Rosso R, Grischke E, Santoro A, et al. Reduced cardiotoxicity and comparable efficacy in a phase III trial of pegylated liposomal doxorubicin HCl (CAELYX/Doxil) versus conventional doxorubicin for first-line treatment of metastatic breast cancer. Ann Oncol 2004; 15(3):440-9.
10. Berry G, Billingham M, Alderman E, Richardson P, Torti F, Lum B, et al. The use of cardiac biopsy to demonstrate reduced cardiotoxicity in AIDS Kaposi's sarcoma patients treated with pegylated liposomal doxorubicin. Ann Oncol 1998; 9(7):711-6.
11. Lotem M, Hubert A, Lyass O, Goldenhersh M A, Ingber A, Peretz T, et al. Skin toxic effects of polyethylene glycol-coated liposomal doxorubicin. Arch Dermatol 2000; 136(12):1475-80.
12. Uziely B, Jeffers S, Isacson R, Kutsch K, Wei-Tsao D, Yehoshua Z, et al. Liposomal doxorubicin: antitumor activity and unique toxicities during two complementary phase I studies. J Clin Oncol 1995; 13(7):1777-85.
13. Lyass O, Uziely B, Ben-Yosef R, Tzemach D, Heshing N I, Lotem M, et al. Correlation of toxicity with pharmacokinetics of pegylated liposomal doxorubicin (Doxil) in metastatic breast carcinoma. Cancer 2000; 89(5):1037-47.
14. Coleman R E, Biganzoli L, Canney P, Dirix L, Mauriac L, Chollet P, et al. A randomised phase II study of two different schedules of pegylated liposomal doxorubicin in metastatic breast cancer (EORTC-10993). Eur J Cancer 2006; 42(7):882-7.
15. Amantea M, Newman M S, Sullivan T M, Forrest A, Working P K. Relationship of dose intensity to the induction of palmar-plantar erythrodysesthia by pegylated liposomal doxorubicin in dogs. Hum Exp Toxicol 1999; 18(1):17-26.
16. Chin D L, Lum B L, Sikic B I. Rapid determination of PEGylated liposomal doxorubicin and its major metabolite in human plasma by ultraviolet-visible high-performance liquid chromatography. J Chromatogr B Analyt Technol Biomed Life Sci 2002; 779(2):259-69.
17. Gabizon A, Catane R, Uziely B, Kaufman B, Safra T, Cohen R, et al. Prolonged circulation time and enhanced accumulation in malignant exudates of doxorubicin encapsulated in polyethylene-glycol coated liposomes. Cancer Res 1994; 54(4):987-92.
18. Vail D M, Amantea M A, Colbern G T, Martin F J, Hilger R A, Working P K. Pegylated liposomal doxorubicin: proof of principle using preclinical animal models and pharmacokinetic studies. Semin Oncol 2004; 31(6 Suppl 13):16-35.
19. Woodle M C, Lasic D D. Sterically stabilized liposomes. Biochim Biophys Acta 1992; 1113(2):171-99.
20. Gabizon A A. Liposome circulation time and tumor targeting: Implications for cancer chemotherapy. Adv Drug Deliv Rev 1995; 16:285-94.
21. Gabizon A, Goren D, Horowitz A T, Tzemach D, Lossos A, Siegal T. Long-circulating liposomes for drug delivery in cancer therapy: a review of biodistribution studies in tumor-bearing animals. Adv Drug Deliv Rev 1997; 24: 337-44.
22. Huang S K, Lee K D, Hong K, Friend D S, Papahadjopoulos D. Microscopic localization of sterically stabilized liposomes in colon carcinoma-bearing mice. Cancer Res 1992; 52(19):5135-43.
23. Storm G, ten Kate M T, Working P K, Bakker-Woudenberg I A. Doxorubicin entrapped in sterically stabilized liposomes: effects on bacterial blood clearance capacity of the mononuclear phagocyte system. Clin Cancer Res 1998; 4(1):111-5.
24. Gordon A N, Fleagle J T, Guthrie D, Parkin D E, Gore M E, Lacave A J. Recurrent epithelial ovarian carcinoma: a randomized phase III study of pegylated liposomal doxorubicin versus topotecan. J Clin Oncol 2001; 19(14):3312-22.
25. Kim R J, Peterson G, Kulp B, Zanotti K M, Markman M. Skin toxicity associated with pegylated liposomal doxorubicin (40 mg/m2) in the treatment of gynecologic cancers. Gynecol Oncol 2005; 97(2):374-8.
26. Colbern G T, Hiller A J, Musterer R S, al. e. Significant increase in antitumor potency of doxorubicin HCl by its encapsulation in pegylated liposomes. J Liposome Res 1999; 9:523-538.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

1. A method of treating malignancies in a human in need of treatment comprising administering to the subject a 60 mg/m² loading dose of a pegylated liposomal doxorubicin (PLD) in an initial cycle, followed by a reduced dose in a second cycle, wherein the second cycle reduced dose is 50% of the initial loading dose, and thereafter one or more maintenance doses in further cycles.

2-8. (canceled)

9. The method of claim 1, wherein the one or more maintenance doses are 45 mg/m².

10. The method of claim 9, wherein the subject is administered two maintenance doses of 45 mg/m².

11. The method of claim 10, wherein the interval between dose cycles is three weeks.

12. The method of claim 10, wherein the interval between dose cycles is four weeks.

13. A method of treating solid tumor malignancies in a human in need of treatment comprising administering to the subject a 60 mg/m² loading dose of a pegylated liposomal doxorubicin (PLD) in an initial cycle, followed by a reduced dose in a second cycle, wherein the second cycle reduced dose is 50% of the initial loading dose, and thereafter one or more maintenance doses in further cycles.

14. The method of claim 13, wherein the malignancies are breast carcinoma.

15. A method of treating hematological malignancies in a human in need of treatment comprising administering to the subject a 60 mg/m² loading dose of a pegylated liposomal doxorubicin (PLD) in an initial cycle, followed by a reduced dose in a second cycle, wherein the second cycle reduced dose is 50% of the initial loading dose, and thereafter one or more maintenance doses in further cycles.

16. The method of claim 15, wherein the malignancies are multiple myeloma.
